(19) United States
(12) Patent Application Publication
(10) Pub. No.: US 2011/0118434 A1
(43) Pub. Date: May 19, 2011

(54) METHOD FOR POLYMERISING GLYCOLIC ACID WITH MICROORGANISMS

(75) Inventors: Philippe Soucaille, Deyme (FR); Wanda Dischert, Vic-Le-Comte (FR)

(73) Assignee: Metabolic Explorer

(21) Appl. No.: 13/003,297

(22) PCT Filed: Jul. 10, 2009
(86) PCT No.: PCT/EP09/58836

§ 371 Date: Jan. 8, 2011

(51) Int. Cl. C12P 7/62; C12N 1/21; C12N 9/00; C12N 9/10; C12N 9/96; C08G 63/06

(52) U.S. Cl. 528/361; 435/135; 435/252.3; 435/183; 435/252.33; 435/193; 435/188

(57) ABSTRACT

The present invention relates to a method for producing and preparing polyglycolate (PGA) from genetically engineered organisms. More specifically, the invention relates to a method comprising two steps; 1) culturing, in a medium containing glycolic acid or not, the microorganism expressing at least one gene encoding an enzyme(s) that converts glycolate into glycolyl-CoA, and a gene encoding polyhydroxyalkanoate (PHA) synthase which uses glycolyl-CoA as a substrate, 2) recovering the polyglycolate polymer.

### FIELD OF THE INVENTION

The present invention relates to a method for making polyglycolic acid polymers called PGA. More specifically, the invention relates to a method comprising the steps of:

cultivating a genetically engineered microorganism with a suitable carbon source, including or not glycolic acid, said microorganism expressing a gene encoding an enzyme that converts glycolate into glycolyl-CoA and at least one gene encoding an enzyme involved in PHA synthesis and

recovering the polyglycolate polymer.

### BACKGROUND OF THE INVENTION

PLA and PGA polymers are biodegradable thermoplastic materials, with a broad range of industrial and biomedical applications (Williams and Peoples, 1996, CHEMTECH 26, 38-44).

These polyesters play important roles not only as industrial plastics but also as medical biopolymers in applications such as drug delivery carriers (Drug delivery and targeting. Nature 392, 5-10 (1998), Langer, R.), biomaterial scaffolds and medical devices (Biodegradable polyesters for medical and ecological applications. Macromol. Rapid

Commum. 21, 117-132 (2000), Ikada, Y. & Tsuji, H.; Sterilization, toxicity, biocompatibility and clinical applications of polylactic acid/polyglycolic acid copolymers. Biomaterials 17, 93-102 (1996), Athanasiou, K. A. et al). PGA is a polyester resin which has good properties: a very high gas impermeability even under 80% humidity, biodegradability, high mechanical strength, and good moldability (Poly (glycolic acid) In polymer data handbook (ed. Mark, J. E.) 566-569 (Oxford University Press, New York, 1999) Lu, L; & Mikos, A. G). This unique combination of properties makes PGA ideally suited for high performance packaging and industrial applications. Today, the targeted application for PGA is multilayer polyethylene terephthalate (PET) bottles for carbonated soft drinks and beer. Since PGA offers a gas barrier 100 times higher that that of PET, it is possible to reduce the amount of PET used in these bottles by more than 20 percent, while maintaining the equivalent barrier against CO2 loss. This bottle redesign has the potential of yielding cost reduction. Perhaps most importantly, PGA's unique hydrolytic properties make it highly compatible with widely practiced industrial PET recycling processes, ensuring the material does not interfere with the purity and quality of recycled PET. In another packaging application, PGA multi-layer designs have been shown to enhance the gas and moisture barrier of bio-based polymers such as polylactic acid (PLA). Through expanded use in biodegradable applications, PGA will further contribute to environmental conservation.

At present, PGA is being prepared by two different chemical routes, either the ring-opening polymerization of cyclic diesters or the polycondensation of 2-hydroxycarboxylic acids. Ring-opening polymerization of cyclic diesters is in three steps: (i) polycondensation of α-hydroxycarboxylic acids, (ii) the synthesis of cyclic diesters by a thermal unzipping reaction and (iii) ring-opening polymerization of the cyclic diester (Preparative Methods of Polymer Chemistry 2^{nd} edition, Interscience Publishers Inc, New York 1963, Sorensen, W. R. & Campbell, T. W.; Controlled Ring-opening Polymerization of Lactide and Glycolide. Chem. Rev. 104, 6147-6176 (2004), Dechy-Cabaret, O. et al.). Alternatively, it is well known that low-molecular-weight PGA can be produced by the direct polycondensation of glycolic acid. The attainment of only low-molecular-weight polymers is largely due to the difficulty in removing water, the by-product during polymerization, which favors depolymerization (Synthesis of polylactides with different molecular weights. Biomaterials 18, 1503-1508 (1997), Hyon, S.-H. et al.). Therefore, ring-opening polymerization of cyclic diesters using coordination initiators is preferred for the synthesis of high-molecular-weight polymers. But this process has disadvantages due to the addition of solvents or chain coupling agents (initiators) which are not easy to remove.

Meanwhile, bacterial polyesters—also referred to as microbial polyesters and polyhydroxyalkanoates, PHAs—are stored as intracellular granules as a result of a metabolic stress upon imbalanced growth due to a limited supply of an essential nutrient and the presence of an excess of a carbon source (Lenz and Marchessault 2005; Lenz 1993; Sudesh et al., 2000; Sudesh and Doi 2005; Steinbüchel and Fiichtenbusch 1998; Steinbüchel and Valentin 1995; Steinbüchel 1991). PHAs are naturally synthesized by a wide range of different Gram-positive and Gram-negative bacteria, as well as by some Archaea. PHAs have attracted considerable attention in recent decades due to similarity in the physical properties of this biopolymer to conventional petrochemical-based polypropylene in terms of their tensile strength and stiffness (Sudesh et al., 2000). Unlike conventional plastics, however, PHAs are biodegradable and recyclable in nature thus, making this class of polymer friendly to the environment.

Two types of PHAs according to the length of the side chain are distinguished.

One type is consisting of short-chain-length hydroxyalkanoic acids, sclPHA, with short alkyl side chains (3-5 carbon atoms) that are produced by Ralstonia eutropha (Lenz and Marchessault 2005).

The second type is consisting of medium-chain-length hydroxyalkanoic acids, mc/PHA, with long alkyl side chains (6-14 carbon atoms) that are produced by Pseudomonas oleovorans and other Pseudomonas (Timm and Steinbüchel, 1990) (Nomura, C. T. & Taguchi, S., 2007; Steinbüchel, A. & Hazer, B., 2007). Although the most well-studied PHA is poly(3-hydroxybutyrate) (PHB), a polymer of 3-hydroxybutyrate (3HB), there are over 150 constituents monomers (Steinbüchel A. Valentin A E. FEMS Microbiol Lett 1995, 128:219-228; Madison L. and Huisman G. Microbiol. and Mol Biol Reviews, 1999, 63:21-53; Rehm B. Biochem J 2003, 376:15-33). This wide variety of monomers yields PHAs with diverse material properties that depend on polymer composition.

The minimal requirements for the synthesis of PHA in a microorganism are source of (>3)-hydroxyalkanoyl-CoA and an appropriate PHA synthase (Gerngross and Martin, PNAS 92:6279-83, 1995). Polyester synthases are key enzymes of polyester biosynthesis and catalyse the conversion of (R)-(>3)-hydroxyacyl-CoA thioesters to polyesters with the concomitant release of CoA. These polyester synthases have been biochemically characterized. An overview of these recent findings is provided in (Rehm, 2003). There are 4 major classes of PHA synthases according to their sequence, their substrate specificity, and their subunit composition (Rhem B. H. A. Biochem J. 2003, 376: 15-33). Owing to the low substrate specificity of PHA synthases that represent the key enzyme for PHA biosynthesis, the variability of bacterial PHAs that can be directly produced by fermentation is extraordinary large. By choosing an appropriate production strain as well as a suitable cultivation conditions and carbon sources, PHA with tailor-made compositions can be produced. There are many examples in the literature showing the production of PHAs by natural producer's organisms like Ralstonia eutropha, Methylbacterium, Pseudomonas and by recombinant bacteria natural producers or not like E. coli (Qi et al., FEMS Microbiol. Lett., 157:155, 1997; Qi et al., FEMS Microbiol. Lett., 167:89, 1998; Langenbach et al., FEMS Microbiol. Lett., 150:303, 1997; Madison L. and Huisman G., 1999; WO 01/55436; U.S. Pat. No. 6,143,952; WO 98/54329; WO 99/61624).

PHA synthase synthesizes PHA using (>3)-hydroxyacyl-CoA as a substrate. Therefore, the first step of polymerization is the obtention of (>3)-hydroxyacyl-coA thioesters, substrates of the synthases. Accordingly, conversion of hydroxy acid to (R)-(>3)-hydroxyacyl-CoA thioesters is an essential step for the biosynthesis of polyesters.

The following enzymes are known as enzymes capable of generating 3-hydroxyacyl-CoA; β-ketothiolase (PhaA), acetoacetyl-CoA reductase (PhaB), cloned from Ralstonia eutropha, 3-hydroxydecanoyl-ACP:CoA transferase (PhaG) cloned from Pseudomonas, (R)-specific enoyl-CoA hydratase (PhaJ) derived from Aeromonas caviae and Pseudomonas aeruginosa (Fukui et al., J. Bacteriol. 180:667, 1998; Tsage et al., FEMS Microbiol. Lett. 184:193, 2000), 3-ketoacyl-ACP reductase (FabG) derived from E. coli and Pseudomonas aeruginosa (Taguchi et al., FEMS Microbiol. Lett. 176:183, 1999; Ren et al., J. Bacteriol. 182:2978, 2000; Park et al., FEMS Microbiol. Lett. 214:217, 2002). Various kinds of PHAs have been synthesized with these enzymes using hydroxyalkanoates hydroxylated at various positions in the carbon chain (mainly the 3, 4, 5, and 6 positions). However, it has been reported that it has a little PHA synthase activity on hydroxyalkanoates which is hydroxylated at the 2-position (Zhan et al., Appl. Microbiol. Biotechnol. 56:131, 2001; Valentin and Steinbüchel, Appl. Microbiol. Biotechnol. 40:699, 1994; Yuan et al., Arch. Biochem. Biophysics. 394:87, 2001).

The propionyl coenzymeA synthetase encoding gene from Salmonella enterica was cloned in 2000 and named PrpE; see for reference (Valentin et al., 2000). Reported substrates of this enzyme are propionate, acetate, 3-hydroxypropionate, and butyrate. This enzyme catalyzes the transformation of these substrates into their corresponding coenzyme A esters. When this enzyme is co-expressed with a PHA synthase from Ralstonia eutropha in a recombinant E. coli, formation of a PHA copolymer is observed.

The acetyl-coA synthetase encoding gene from Escherichia coli was cloned in 2006 and named acs; see for reference (Lin et al., 2006). When overexpressed in E. coli, this enzyme reduces the acetate accumulation into the microorganism, by transforming said acetate into acetyl-coA.

Although over 150 different monomers have been incorporated into PHAs in organisms, the production of biosynthetic polyglycolide PGA has never been reported, because a hydroalkanoate, such as glycolate hydroxylated at the 2-position carbon, is not a suitable substrate for PHA synthase.

Two patent applications describe the incorporation of 2-hydroxyacid monomers in polymers by the action of a PHA synthase in living cells.

US 2007/0277268 (Cho et al.) relates the bioproduction of polylactate (PLA) or its copolymers by cells or plants.

WO 2004/038030 (Martin et al.) shows the formation of co-polymers containing monomers of glycolyl-CoA and at least one other monomer selected from the group consisting of 3-hydroxybutyric acid, 3-hydroxypropionic acid, 3-hydroxyvaleric acid, etc In this case, the substrate glycolyl-CoA is obtained via the 4-hydroxybutyryl-CoA molecule and a reaction requiring FadE, AtoB and thiolase II.

Up to now, the available prior art documents have never reported a process for the production of a homopolymer of glycolic acid (PGA) by fermentation of a microorganism.

Here, inventors have developed a method to produce high-molecular-weight PGA using microorganisms. As disclosed herein, the inventors describe that polyglycolic acid homopolymers is produced by culturing recombinant microorganisms transformed with a PHA synthase gene and a gene encoding an enzyme that converts glycolate into glycolyl-CoA, in a production medium containing a suitable carbon source.

### DESCRIPTION OF THE INVENTION

It is an object of the present invention to provide a method for the biosynthesis of PGA, a homopolymer of glycolic acid.

This method is based on the use of a recombinant microorganism, expressing:
1. a gene encoding for an heterologous polyhydroxyalkanoate (PHA) synthase, and
2. at least one gene encoding for an enzyme(s) transforming the glycolic acid into glycolyl-coA.

Other objects of the invention are a biosynthetic PGA such as obtained by the process according to the invention, and a microorganism expressing genes for biosynthesis of PGA according to the invention.

### BRIEF DESCRIPTION OF DRAWINGS

FIG. 1 shows the successive reactions for production of polyglycolic acid polymer, PGA. The first reaction is the formation of glycolyl-CoA, substrate of the second reaction of polymerisation catalyzed by the PHA synthase.

FIG. 2 is a schematic diagram showing the pathway for synthesizing polyglycolate using cells cultivated on a medium containing glucose plus glycolate.

FIG. 3 is a schematic diagram showing the pathway for synthesizing polyglycolate using cells cultivated on a medium containing glucose without any exogenous glycolate.

FIG. 4 represents the microscopic observation (X100) of strain AG1122 grown in Erlenmeyer flask in LB+glucose. Optical microscope AVANTEC 3804921.

FIG. 5 represents the microscopic observation (X100) of strain AG1122 grown in fermentor in LB+glucose. Optical microscope AVANTEC 3804921.

FIG. 6 represents the microscopic observation (X100) of strain AG1327 grown in Erlenmeyer flask in modified M9+glucose. Optical microscope AVANTEC 3804921

FIG. 7 represents LC-MS chromatogram of the reaction with glycolate on crude cell extract of the strain AG1354

FIG. 8 represents LC-MS chromatogram of the reaction with glycolate on the pure protein PrpEst.

### DETAILED DESCRIPTION OF THE INVENTION

The present invention is related to a method for obtaining the polymerisation of glycolic acid into PGA with a microorganism, comprising the steps of:
cultivating a microorganism expressing a gene encoding for an heterologous polyhydroxyalkanoate (PHA) synthase, in a medium comprising a carbon source,
and recovering the polymerised glycolic acid (PGA),
   wherein the microorganism also expresses at least one gene encoding for an enzyme(s) transforming the glycolic acid into glycolyl-coA.

The term “polymerization” or “homopolymerization” means a chemical reaction in which the molecules of a monomer are linked together to form large molecules whose molecular weight is a multiple of that of the original substance. When two or more different monomers are involved, the process is called copolymerization or heteropolymerization.

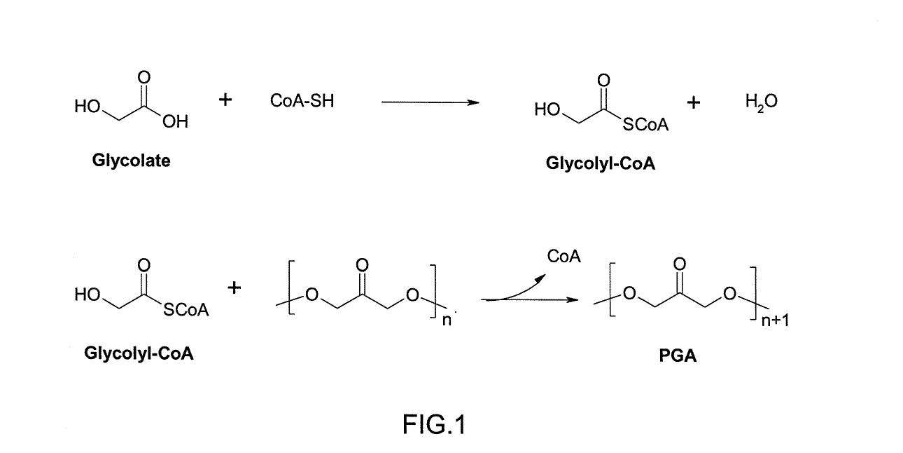

‘PGA’ designates the polyglycolic acid, also called polyglycolate consisting of glycolic acid-recurring unit represented on the FIG. 1 and by the formula I below:

—(—O—CH₂—CO—)—

PGA is a homopolymer comprising at least at 55 wt. % of the above-mentioned glycolic acid-recurring unit (also called glycolate). The content of the above-mentioned glycolic acid-recurring unit in the PGA resin is at least 55 wt. %, preferably at least 70 wt. %, further preferably 90 wt. %.

PGA may preferably have a weight-average molecular weight in a range of 10,000-600,000 Daltons according to GPC measurement using hexafluoroisopropanol solvent. Weight-average molecular weights of 150,000-300,000 Daltons are further preferred.

According to the invention the terms ‘culture’ or ‘fermentation’ are used interchangeably to denote the growth of bacteria on an appropriate growth medium containing a carbon source.

The sentence “recovering the polymerised glycolic acid from the culture medium” designates the action of recovering PGA such as well known by the man skilled in the art. In particular, after the producing cells are collected by centrifugation and freeze-dried, polymer substance accumulated in the strains is recovered using solvents such as HexaFluorolsoPropanol (HFIP), TetraHydroFurane (THF), DiMethylSulfOxyde or

Chlororforme (Lageveen et al., 1988; Amara et al., 2002). PGA is extracted from lyophilized cells using one of the solvents mentioned above, most preferentially using HFIP solvent and subsequently precipitated in ethanol or methanol. The precipitate is obtained by centrifugation, dissolved in chloroform and precipitated again in order to highly purified PGA. Polymers are further analyzed by NMR.

The term “microorganism” designates a bacterium, yeast or fungus. Preferentially, the microorganism is selected among Enterobacteriaceae, Bacillaceae, Streptomycetaceae and Corynebacteriaceae. More preferentially, the microorganism is a species of Escherichia, Klebsiella, Pantoea, Salmonella or Corynebacterium. Even more preferentially, the microorganism is Escherichia coli.

The term ‘carbon source’ according to the present invention denotes any source of carbon that can be used by those skilled in the art to support the normal growth of a microorganism, which can be hexoses (such as glucose, galactose or lactose), pentoses, monosaccharides, disaccharides (such as sucrose, cellobiose or maltose), oligosaccharides, molasses, starch or its derivatives, hemicelluloses, glycerol and combinations thereof. An especially preferred simple carbon source is glucose. Another preferred simple carbon source is sucrose.

The term “an enzyme transforming the glycolic acid into glycolyl-CoA” designates an enzyme able to activate glycolic acid molecules into glycolyl-CoA, substrate for the PHA synthase in the polymerization process.

According to a first aspect of the invention, the glycolic acid is produced by the same microorganism expressing genes encoding a PHA synthase and at least one enzyme transforming the glycolic acid into glycolyl-CoA. Microorganisms producing high level of glycolic acid by fermentation from a renewable source of carbon have been previously described; see in particular WO 2007/140816 and WO 2007/141316.

It would be also advantageous to reduce the exportation of glycolic acid from this glycolic acid producing microorganism. The man skilled in the art knows numerous means to obtain such reduction of transport of a specific metabolite, in particular reducing or inhibiting the activity and/or the expression of a transport protein, able to export glycolic acid from the microorganism to the medium.

According to a second aspect of the invention, the glycolic acid is provided to the microorganism exogenously in the culture medium.

In particular, an amount of at least 2 grams/Liter of glycolic acid is added in the culture medium, preferentially at least 10 g/L. The man skilled in the art will adjust the dose in a way to avoid the toxicity of high concentrations of glycolic acid, such as 30 g/L. As described previously, the exportation of glycolic acid may be reduced or even totally prevented in the microorganism according to the invention. It would be also advantageous to improve the import of glycolic acid present in the culture media. The man skilled in the art knows numerous means to obtain such improvement of transport of a specific metabolite, in particular increasing the activity and/or the expression of a permease protein, able to import glycolic acid from the medium to the microorganism. In particular, it would be advantageous to overexpress the genes glcA, lldP and yjcG encoding glycolate importers (Nunez, F. et al., 2001 Microbiology, 147, 1069-1077; Nunez, F. et al., 2002 Biochem. And Biophysical research communications 290, 824-829; Gimenez, R. et al., 2003 J. of Bacteriol. 185, 21, 6448-6455).

In a preferred aspect of the invention, the enzyme transforming the glycolic acid into glycolyl-CoA is chosen among:
acyl-CoA synthetases or acyl-CoA transferases,
phosphotransbutyrylase associated to butyrate kinase.
   Acyl-CoA transferases found in anaerobic bacteria are known to catalyze the formation of short- to medium-chain-length CoA-thioesters (Mack, M. and Buckel, W., 1997).

In a first aspect of the invention, the enzyme transforming the glycolic acid into glycolyl-CoA is chosen among genes belonging to Enterobacteriaceae species and most preferred is:
a propionyl coenzyme A synthetase from Escherichia coli or Salmonella Thyphimurium encoded by the gene prpE; or
the acetyl-CoA transferase from E. coli encoded by the gene acs.

In a second embodiment of the invention, the phophotransbutyrylase is encoded by the gene ptb and the butyrate kinase is encoded by the gene buk.

The terms “encoding” or “coding” refer to the process by which a polynucleotide, through the mechanisms of transcription and translation, produces an amino-acid sequence. This process is allowed by the genetic code, which is the relation between the sequence of bases in DNA and the sequence of amino-acids in proteins. One major feature of the genetic code is to be degenerate, meaning that one amino-acid can be coded by more than one triplet of bases (one “codon”). The direct consequence is that the same amino-acid sequence can be encoded by different polynucleotides. It is well known from the man skilled in the art that the use of codons can vary according to the organisms. Among the codons coding for the same amino-acid, some can be used preferentially by a given microorganism. It can thus be of interest to design a polynucleotide adapted to the codon usage of a particular microorganism in order to optimize the expression of the corresponding protein in this organism.

In the description of the present invention, genes and proteins are identified using the denominations of the corresponding genes in E. coli. However, and unless specified otherwise, use of these denominations has a more general meaning according to the invention and covers all the corresponding genes and proteins in other organisms, more particularly microorganisms.

PFAM (protein families database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG/ are obtained by comparing protein sequences from 66 fully sequenced genomes representing 30 major phylogenic lines. Each COG is defined from at least three lines, which permits the identification of former conserved domains.

The means of identifying homologous sequences and their percentage homologies are well known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.p1), with the default parameters indicated on those websites.

Using the references given in GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well known to those skilled in the art, and are claimed, for example, in Sambrook et al. (1989 Molecular Cloning: a Laboratory Manual. 2^{nd} ed. Cold Spring Harbor Lab., Cold Spring Harbor, N.Y.).

The present invention is also related to an expression cassette comprising a polynucleotide encoding an enzyme transforming the glycolic acid into glycolyl-CoA under the control of regulatory elements functional in a host microorganism.

The term “expression” refers to the transcription and translation of a gene sequence leading to the generation of the corresponding protein, product of the gene.

In a preferred aspect of the invention, the gene(s) encoding for the enzyme(s) transforming the glycolic acid into glycolyl-CoA is(are) overexpressed into the microorganism.

The terms “increased expression” “enhanced expression” or “overexpression” are used interchangeably in the text and have similar meaning, i.e. that the transcription and translation of the gene is increased compared to a non-recombinant microorgansim, leading to an increased amount of enzyme into the cell.

To increase the expression of a gene, the expert in the field knows different ways to manipulate genes expression. In particular, the gene may be expressed using promoters with different strength, which may be inducible. These promoters may be homologous or heterologous. The man skilled in the art knows which promoters are the most convenient, for example, promoters Ptrc, Ptac, Plac or the lambda promoter cI are widely used.

In an embodiment of the invention, the gene(s) may be expressed by a plasmid or vector introduced into the microorganism. Said microorganism is then said a “host microorganism”, referring to a microorganism able to receive foreign or heterologous genes or extra copies of its own genes and able to express those genes to produce an active protein product.

The term “transformation” refers to the introduction of new genes or extra copies of existing genes into a host organism. As an example, in E. coli, a method for transferring DNA into a host organism is electroporation.

The term “transformation vector” refers to any vehicle used to introduce a polynucleotide in a host organism. Such vehicle can be for example a plasmid, a phage or other elements known from the expert in the art according to the organism used. The transformation vector usually contains in addition to the polynucleotide or the expression cassette other elements to facilitate the transformation of a particular host cell. An expression vector comprises an expression cassette allowing the suitable expression of the gene borne by the cassette and additional elements allowing the replication of the vector into the host organism. An expression vector can be present at a single copy in the host organism or at multiple copies. The man skilled in the art knows different types of plasmids that differ with respect to their origin of replication and thus their copy number in the cell. They may be present as 1-5 copies, about 20 or up to 500 copies, corresponding to low copy number plasmids with tight replication (pSC101, RK2), low copy number plasmids (pACYC, pRSF1010) or high copy number plasmids (pSK bluescript II).

The present invention provides a transformation vector comprising a gene encoding for an enzyme transforming the glycolic acid into glycolyl-coA.

In another embodiment of the invention, said gene(s) may be integrated into the chromosome of the microorganism. There may be one or several copies of the gene that can be introduced into the genome of an organism, by methods of recombination well known by the man skilled in the art.

Another mean to obtain an overexpression of the genes is to modify the expression or regulation of the elements stabilizing the corresponding messenger RNA (Carrier et al. Biotechnol Bioeng. 59:666-72, 1998) if translation of the mRNA is optimized, then the amount of available enzyme is increased.

The recombinant microorganism used in the invention also expresses a gene encoding for a polyhydroxyalkanoate synthase. Four major classes of PHA can be distinguished (Rhem, B., 2003). Class I and Class II PHA synthases comprise enzymes consisting of only one type of subunit (PhaC). According to their in vivo and in vitro specificity, class I PHA synthases (e.g. in Ralstonia eutropha) preferentially utilize CoA-thioester of various hydroxy fatty acids comprising 3 to 5 carbons atoms, whereas class II PHA synthases (e.g. in Pseudomonas aeruginosa) preferentially utilize CoA-thioester of various hydroxy fatty acids comprising 6 to 14 carbon atoms. Class III synthases (e.g. in Allochromatium vinosum) comprises enzymes consisting of two different types of subunits: the PhaC and the PhaE subunits. These PHA synthases prefer CoA-thioesters of hydroxy fatty acids comprising 3 to 5 carbons atoms. Class IV PHA synthases (e.g. in Bacillus megaterium) resemble the class III PHA synthases, but PhaE is replaced by PhaR.

In a specific embodiment of the invention, the gene encoding the heterologous PHA synthase is chosen among phaC, phaEC or phaCR, preferentially among phaC and phaEC and most preferentially the gene selected is phaC encoding an enzyme of Class I PHA synthases. As previously exposed, use of these denominations ‘phaC’, ‘phaEC’ and ‘phaCR’ cover all the corresponding genes and proteins in other organisms, more particularly microorganisms. Indeed, using the references given in GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. All equivalent genes are incorporated herein by reference.

Preferentially, said gene encoding a heterologous PHA synthase is overexpressed. As previously described, the overexpression of a gene may be obtained by different ways known by the man skilled in the art; the gene may be expressed by an expression vector introduced into the microorganism, or be integrated into the chromosome of said microorganism.

In a preferred embodiment of the invention, the recombinant microorganism used in the method also expresses a PhaR/PhaP regulatory system, particularly the microorganism expresses genes phaP and phaR from W. eutropha encoding respectively for a phasin and its transcriptional expression regulator. The phasin protein, PhaP is likely to be involved in maintenance of the optimal intracellular environment of PHA synthesis and provides guidance during the process of granule formation (Wieczorek, R. et al. 1995). The PhaR homologs have been investigated both in vitro and in vivo (Wieczorek R. et al. 1995 and York G. M. et al. 2002) and is proposed to function as a regulator of the phaP transcription.

Use of these denominations ‘phaP’ and ‘phaR’ cover all the corresponding genes and proteins in other microorganisms.

The invention is also relative to a polymerised glycolic acid (PGA) obtained by the method according to the invention.

The main advantage of the invention is to produce PGA in an easier and cheaper way than the chemical one that necessitates the use of glycolide, a compound difficult to produce from glycolic acid.

The invention is also relative to a microorganism expressing genes encoding for a heterologous PHA synthase and at least one enzyme transforming the glycolic acid into glycolyl-CoA.

Preferentially, said microorganism is an Enterobacteriaceae, more preferentially an Escherichia coli.

### EXAMPLES

TABLE 1
sequences of the oligonucleotides used in the constructions described below.

| Gene | Name | Sequence | Objective |
|---|---|---|---|
| prpE F | SEQ ID NO 1 | tctagaggatccaagttcaacaggagagcattatg | overexpression |
| prpE R | SEQ ID NO 2 | ggatccgctagccctaggtacgtactactcttccatcgcctggc | overexpression |
| prpEst F | SEQ ID NO 3 | cgatttaattaatctagaggcgtaagttctaaggaggtatattatgtc | overexpression |
| | | ttttagcgaattttatcagcg | |
| prpEst R | SEQ ID NO 4 | tacgcctagggctagcctattcttcgatcgcctggcg | overexpression |
| acs F | SEQ ID NO 5 | tctagaagatctcctacaaggagaacaaaagcatg | overexpression |
| acs R | SEQ ID NO 6 | agatctgctagccctaggtacgtattacgatggcatcgcgatag | overexpression |

Ptrc01 promoter with operator and RBS sequence
SEQ ID N^{o} 7:
gagctgttgacaattaatcatccggctcgtataatgtgtggaattgtgag
cggataacaattTACGTAtaaggaggtatatt
In capital letter: restriction site SnaBI.

### Example 1

### Construction of Recombinant Vectors Containing a Gene Encoding acyl-CoA synthetase

Construction of pSCB-acs, pSCB-prpE and pSCB-prpEst

Two proteins are used to transform glycolic acid in glycolyl-CoA, either a propionyl-CoA synthetase encodes by prpE (from E. coli or from S. thyphimurium) or an acetyl-CoA synthetase encodes by acs. Each gene is co-expressed in the cell with the gene phaC1 from Ralstonia eutropha encoding the PHA synthase.

To amplify acs and prpE genes, PCR are carried out using chromosomal DNA of Escherichia coli as template and the above primers (cf. table 1), named asc F and acs R for acs amplification and prpE F and prpE R for prpE amplification.

The PCR fragment of acs is cloned into the vector pSCB (Stratagene Blunt PCR Cloning Kit CAT 240207-5) resulting in plasmid pSCB-acs.

The PCR fragment of prpE is cloned into the vector pSCB resulting in plasmid pSCB-prpE.

To amplify the gene prpE from Salmonella enterica thyphimurium, a PCR is carried out using plasmid pPRP45 (from Alexander R Horswill, Jorge C Escalante-Semerena “Characterization of the Propionyl-CoA Synthetase (PrpE) Enzyme of Salmonella enterica: Residue Lys592 Is Required for Propionyl-AMP Synthesis”) as template and the above primers (cf. table 1), named prpEst F and prpEst R for prpEst amplification.

The PCR fragment of prpEst is cloned into the vector pSCB (Stratagene Blunt PCR Cloning Kit CAT 240207-5) resulting in plasmid pSCB-prpEst.

### Example 2

### Construction of Recombinant Vectors Containing Genes Encoding PHA Synthase and acyl-CoA Synthetase

Construction of pMK-Ptrc01/OP01/RBS01-phaClre-TT02

The plasmid carrying the gene phaC1 from Ralstonia eutropha is provided by a company that synthesized the gene with an optimized sequence to get the best transcription rate in E. coli.

The relative frequency of codon use varies widely depending on the organism and organelle. Many design programs for synthetic protein coding sequences allow the choice of organism. The codon usage database has codon usage statistics for many common and sequenced organisms like E. coli.

The synthetic gene phaC1 encoding the PHA synthase is provided ready to use by the company. The gene is cloned under a Ptrc01 promoter with operator and RBS sequences (SEQ No 1) located upstream the gene, and a terminator sequence located downstream phaClre, leading to the plasmid pMK-Ptrc01/OP01/RBS01-phaClre-TT02.

Construction of pUC19-Ptrc01/OP01/RBS01-phaClre-TT02 and pBBR1MCS5-Ptrc01/OP01/RBS01-phaClre-TT02

Plasmid pMK-Ptrc01/OP01/RBS01-phaClre-TT02 is digested with HindIII and BamHI and the resulting DNA fragment comprising Ptrc01/OP01/RBS01-phaClre-TT02 is cloned into the vector pBBR1MCS5 cut by the same restriction enzymes. The resulting plasmid is named pBBR1MCS5-Ptrc01/OP01/RBS01-phaClre-TT02.

Plasmid pMK-Ptrc01/OP01/RBS01-phaClre-TT02 is digested with HindIII and BamHI and the resulting DNA fragment comprising Ptrc01/OP01/RBS01-phaClre-TT02 is cloned into the vector pUC19 cut by the same restriction enzymes. The resulting plasmid is named pUC19-Ptrc01/OP01/RBS01-phaClre-TT02.

Construction of pMK-Ptrc01/OP01/RBS01-phaClre-acs-TT02, pMK-Ptrc01/OP01/RBS01-phaClre-prpE-TT02

Plasmids pSCB-acs and pSCB-prpE are digested with XbaI and NheI and the resulting DNA fragments comprising either acs or prpE are cloned into the vector pMK-Ptrc01/OP01/RBS01-phaClre-TT02 cut by the same restriction enzymes. The resulting plasmids are named pMK-Ptrc01/OP01/RBS01-phaClre-acs-TT02 and pMK-Ptrc01/OP01/RBS01-phaClre-prpE-TT02.

Construction of pBBR1MCS5-Ptrc01/OP01/RBS01-phaClre-prpEst-TT02 and pUC19-Ptrc01/OP01/RBS01-phaClre-prpEst-TT02

Plasmid pSCB-prpEst is digested with PacI and NheI and the resulting DNA fragment comprising prpEst is cloned into the vector pBBR1MCS5-Ptrc01/OP01/RBS01-phaClre-TT02 cut by the same restriction enzymes. The resulting plasmid is named pBBR1MCS5-Ptrc01/OP01/RBS01-phaClre-prpEst-TT02.

Plasmid pSCB-prpEst is digested with PacI and NheI and the resulting DNA fragment comprising prpEst is cloned into the vector pUC19-Ptrc01/OP01/RBS01-phaClre-TT02 cut by the same restriction enzymes. The resulting plasmid is named pUC19-Ptrc01/OP01/RBS01-phaClre-prpEst-TT02.

### Example 3

### Construction of Recombinant E. coli Strains Producing PGA when Cultivated in Presence of Glycolate and Preparation of Polyglycolate Polymer

Vector pMK-Ptrc01/OP01/RBS01-phaClre-acs-TT02 and pMK-Ptrc01/OP01/RBS01-phaClre-prpE-TT02 are introduced by electroporation into an E. coli MG1655 wild-type strain, leading to strains MG1655 (pMK-Ptrc01/OP01/RBS01-phaClre-acs-TT02) and MG1655 (pMK-Ptrc01/OP01/RBS01-phaClre-prpE-TT02) respectively.

Vector pBBRMCS5-Ptrc01/OP01/RBS01-phaClre-prpEst-TT02 and pUC19-Ptrc01/OP01/RBS01-phaClre-prpEst-TT02 are introduced by electroporation into an E. coli MG1655 wild-type strain, leading to strains MG1655 (pBBRMCS5-Ptrc01/OP01/RBS01-phaClre-acs-TT02) and MG1655 (pUC19-Ptrc01/OP01/RBS01-phaClre-prpE-TT02) respectively.

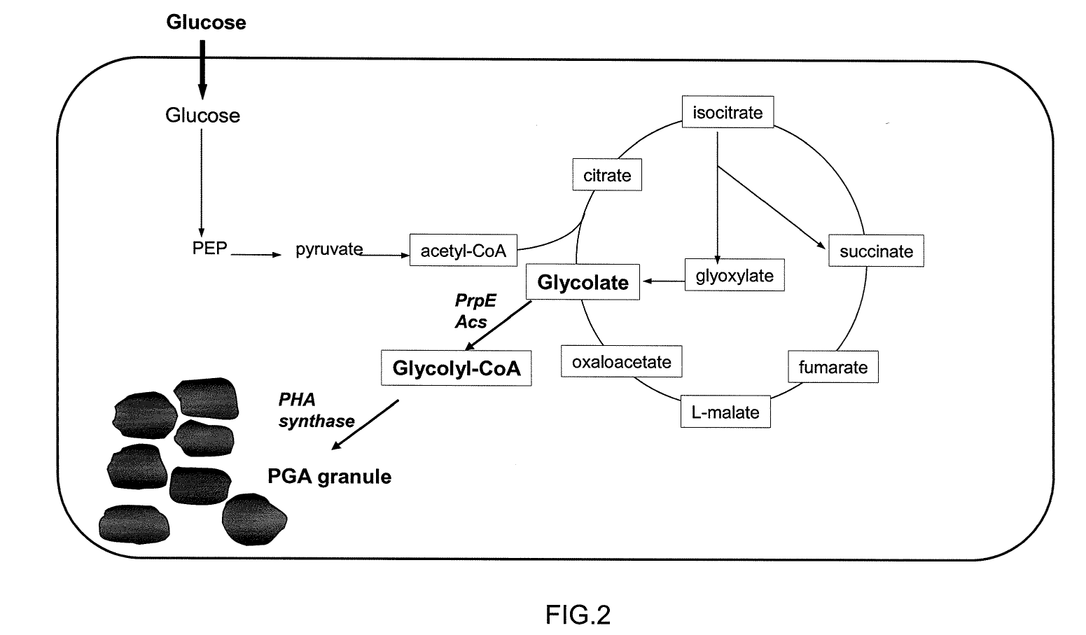

The resulting strains (FIG. 2) are cultured in LB or MM media containing around 5 g/L of glycolate (details of conditions in following examples), followed by centrifugation to recover the strains. The recovered strains are freeze-dried to recover polymer substance accumulated in the cells using solvents as hexafluoroisopropanol or chloroform. To confirm that the obtained polymer is polyglycolate, NMR analyses are done on the recovered polymer substance.

Construction of Recombinant E. coli Strains Producing PGA when Cultivated on Glucose Only and Preparation of Polyglycolate Polymer

The strains genetically engineered to produce glycolic acid from glucose as a carbon source are disclosed in patents WO 2007/141316 A, WO 2007/140816 A, U.S. 61/162,712 and EP 09155971, 6. The strains are used herein for introduction of plasmids allowing production of PGA from glucose only.

Vector pMK-Ptrc01/OP01/RBS01-phaClre-prpE-TT02 and pMK-Ptrc01/OP01/RBS01-phaClre-acs-TT02 are introduced by electroporation into an E. coli strain genetically modified to produce glycolic acid.

Vector pBBRMCS5-Ptrc01/OP01/RBS01-phaClre-prpEst-TT02 and pUC19-Ptrc01/OP01/RBS01-phaClre-prpEst-TT02 are introduced by electroporation into an E. coli strain genetically modified to produce glycolic acid.

### Example 4

### Fermentation of a Strain Producing POLYglycolic Acid Polymer from Glucose in Erlenmeyer Flasks

The production of PGA by fermentation was done with the strain AG1122 having the following genotype (MG1655 Ptrc50/RBSB/TTG-icd::Cm ΔaceB Δgcl ΔglcDEFGB ΔaldA ΔiclR Δedd+eda ΔpoxB ΔackA+pta (pME101-ycdW-TT07-PaceA-aceA-TT01) (pMK-Ptrc01/OP01/RBS01-phaClre-prpE-TT02))

The production of PGA in intracellular was done in 500 ml baffled Erlenmeyer flask (this example) and in batch fermentor (Example 5).

The strain AG1122 was grown in 500 ml baffled Erlenmeyer flask cultures using LB broth (Bertani, 1951, J. Bacteriol. 62:293-300) or a modified M9 medium (Anderson, 1946, Proc. Natl. Acad. Sci. USA 32:120-128) supplemented with 12.5 g/l glucose itself containing 5 g/l MOPS and 5 g/L of glucose. The pH of the medium was then adjusted to pH6.8. The antibiotics Spectinomycin and kanamycin were added to a final concentration of 50 mg/l. An overnight preculture was used to inoculate a 50 ml culture to an OD_{600 nm} of 0.3. The cultures were kept on a shaker at 37° C. and 200 rpm until the glucose in the culture medium was exhausted. Polyglycolic acid production was followed by microscopic observations with an optical microscope of Avantec 3804921.

A picture of the cells after several hours of growth is presented on FIG. 1. The white zones in the cells correspond to granules of polymer.

### Example 5

### Fermentation of a Strain Producing POLYglycolic Acid Polymer from Glucose in Batch Fermentor

The production of PGA by the strain AG1122 (MG1655 Ptrc50/RBSB/TTG-icd::Cm ΔaceB Δgcl ΔglcDEFGB ΔaldA ΔiclR Δedd+eda ΔpoxB ΔackA+pta (pME101-ycdW-TT07-PaceA-aceA-TT01) (pMK-Ptrc01/OP01/RBS01-phaClre-prpE-TT02)) was assessed in production conditions in a 600 ml fermentor using a fed batch protocol.

A unique preculture was carried out in 21 Erlenmeyer flask filled with 200 ml of LB broth (Bertani, 1951, J. Bacteriol. 62:293-300) that was supplemented with 12.5 g/l glucose at 37° C. during 24 hours. This preculture was used for inoculation of the fermentor.

The fermentor filled with 400 ml of LB broth supplemented with 20 g/l of glucose and 50 mg/l of spectinomycin and kanamycin was inoculated at an initial optical density of 1.5. The culture was carried out at 37° C. with agitation and aeration adjusted to maintain the dissolved oxygen above 30% saturation. The pH was adjusted at 6.8 with addition of base. The culture was carried out in a batch mode for 24 hours or until OD₆₀₀ₙₘ>10.

Polyglycolic acid polymer production was followed by microscopic observations. A picture of the strain under microscope is presented on FIG. 2.

The final titer of glycolic acid obtained in that culture was 1.5 g/l (supernatant analyzed by HPLC using a Biorad HPX 97H column for the separation and a refractometer for the detection).

The same fermentation was done in modified M9 medium supplemented with glucose. The production was slower than in the LB broth.

### Example 6

### Fermentation of a Strain Producing POLYglycolic Acid Polymer by Bioconversion of Glycolic Acid in Erlenmeyer Flasks

The bioconversion of glycolic acid into PGA was performed with the strain AG1327 (MG1655 (pUC19-Ptrc01/OP01/RBS01-phaClre-prpEst-TT02))

The bioconversion by AG1327 was assessed in 500 ml baffled Erlenmeyer flask cultures using modified M9 medium that was supplemented with 5 g/l MOPS, 5 g/l glucose and 5 g/L glycolic acid and adjusted at pH 6.8. A supply of LB broth at 10% v/v was also added in order to enhance biomass growth. Ampicillin or carbenicillin was added at a concentration of 100 mg/l. An overnight preculture was used to inoculate a 50 ml culture to an OD_{600 nm} of 0.3. The cultures were kept on a shaker at 30° C. and 200 rpm and polymer production was followed by microscopic observations.

At the end of the culture, glucose and glycolic acid were analyzed by HPLC using a Biorad HPX 97H column for the separation and a refractometer for the detection.

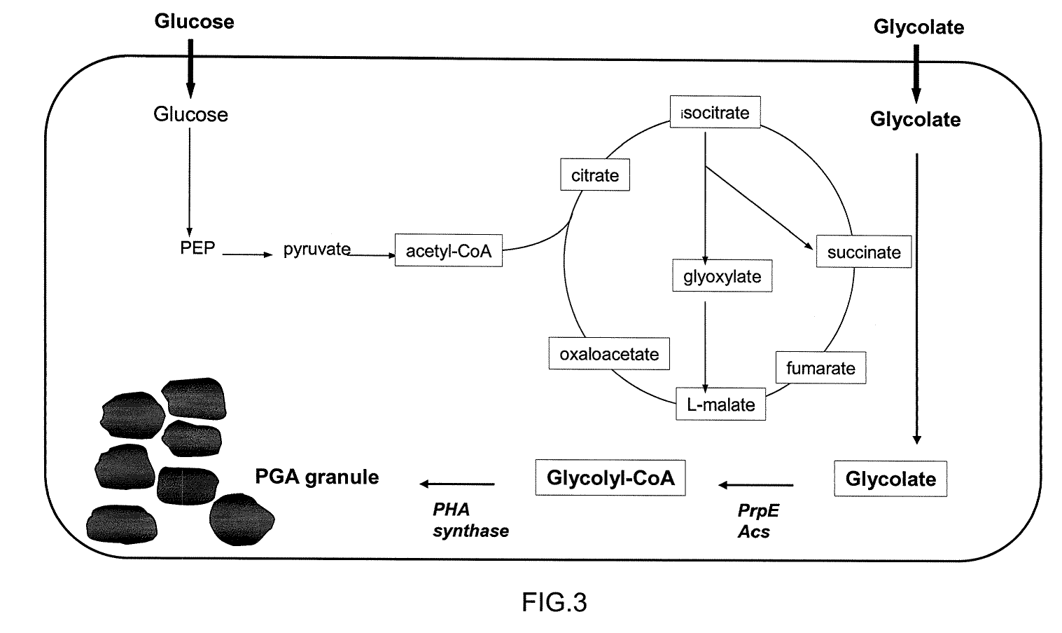

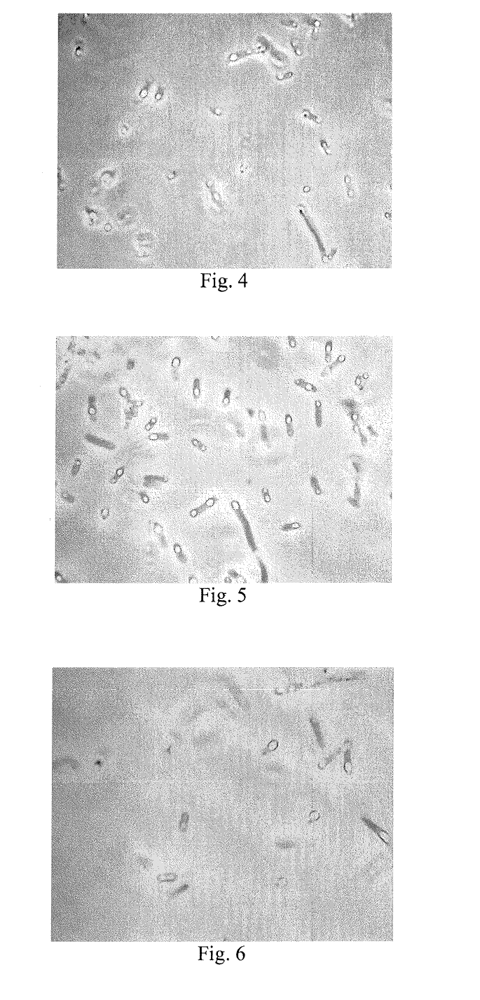

Granules of polymers were observed in presence of glycolic acid and not in the control culture, without addition of glycolic acid (FIG. 3).

Although the present invention has been described in detail with reference to the specific features, it will be apparent to those skilled in the art that this description is only for a preferred embodiment and does not limit the scope of the present invention. Thus, the substantial scope of the present invention will be defined by appended claims and equivalents thereof.

### Example 7

### Conversion of Glycolic Acid into glycolyl-CoA by the Propionyl-CoA Synthetase from Salmonella typhimurium

Construction of the Recombinant BL21 (DE3) (pLysS) (pPAL-prpEst) Strain

To amplify the gene prpE from Salmonella enterica thyphimurium, a PCR is carried out using plasmid pPRP45 as template and the primers named pPAL-prpEst R and pPAL-prpEst F

pPAL-prpEst R
(SEQ ID NO 8)
CGAATTCCTATTCTTCGATCGCCTGGCG
pPAL-prpEst F
(SEQ ID NO 9)
CCCAAGCTTTGATGTCTTTTAGCGAATTTTATCAGCG

PCR product is digested with HindIII and EcoRI and cloned into the vector pPAL7 (Profinity eXact pPAL7 Vector Biorad) cut by the same restriction enzymes. The resulting plasmid is named pPAL-prpEst.

Vector pPAL-prpEst is introduced into E. coli BL21 (DE3) (pLysS) chemically competent cells, leading to strain BL21 (DE3) (pLysS) (pPAL-prpEst) named AG1354.

### Overproduction of the Propionyl-CoA Synthetase, PrpEst

The overproduction of the protein PrpEst is done in a 2 L Erlenmeyer flask.

A unique preculture is carried out in 500 mL Erlenmeyer flask filled with 50 ml of LB broth (Bertani, 1951, J. Bacteria 62:293-300) that is supplemented with 5 g/l glucose, 100 ppm of ampicilline and 1 g/L MgSO₄. The preculture is cultivated at 37° C., 200 rpm until OD₆₀₀=0.5 and then used for inoculation of the 2 L flask filled with 500 mL of LB supplemented with 5 g/l glucose, 100 ppm of ampicilline and 1 g/L MgSO₄. The culture is first carried out at 37° C. and 200 rpm until OD₆₀₀ is 0.6-0.8, and in a second step moved at 25° C. before the induction with 500 μM IPTG. The culture is stopped when the OD₆₀₀ is around 4. Cells are centrifuged at 7000 rpm, 10 minutes at 4° C., and then washed with phosphate buffer before to be stored at −20° C.

### Purification of the Protein PrpEst

The cells (45 mg) are lysed by sonication and cell debris are removed by centrifugation at 12000 g (4° C.) for 30 min. The protein is purified from the crude cell-extract by affinity on a Profinity column (BIORAD, Bio-Scale Mini Profinity exact cartridge) according to the protocol recommended by the manufacturer. The Tag is removed from the protein by cleavage with 100 mM fluoride at room temperature for 30 min. The elution buffer is exchanged by dialysis against a solution composed of 100 mM potassium phosphate, 150 mM NaCl and 10% glycerol.

The Bradford protein assay is used to measure protein concentration (i.e. 0.23 μg/μL for 45 mg of dried weight).

Detection of glycolyl-CoA by LC-MS

The activity of PrpE on glycolate is measured by LC-MS (Applied/DIONEX), by detection of the resulting molecule, the glycolyl-CoA (chemical features on scheme 1). The reaction mixture (250 μL) contains 75 mM of potassium phosphate buffer (pH 7.5), 1.5 mM ATP, 0.75 mM CoA and either 10 to 40 μg of crude cell-extract or 9 μg of purified protein. Reaction mixtures were started with different concentrations of glycolate (20, 40 and 100 mM). Samples were incubated at 37° C. for 30 min before to be injected on the LC-MS machine (25 μL or 75 μL of the reaction were loaded). Three reaction mixtures were prepared as controls; 1) without CoA, 2) without enzyme or 3) without glycolate.

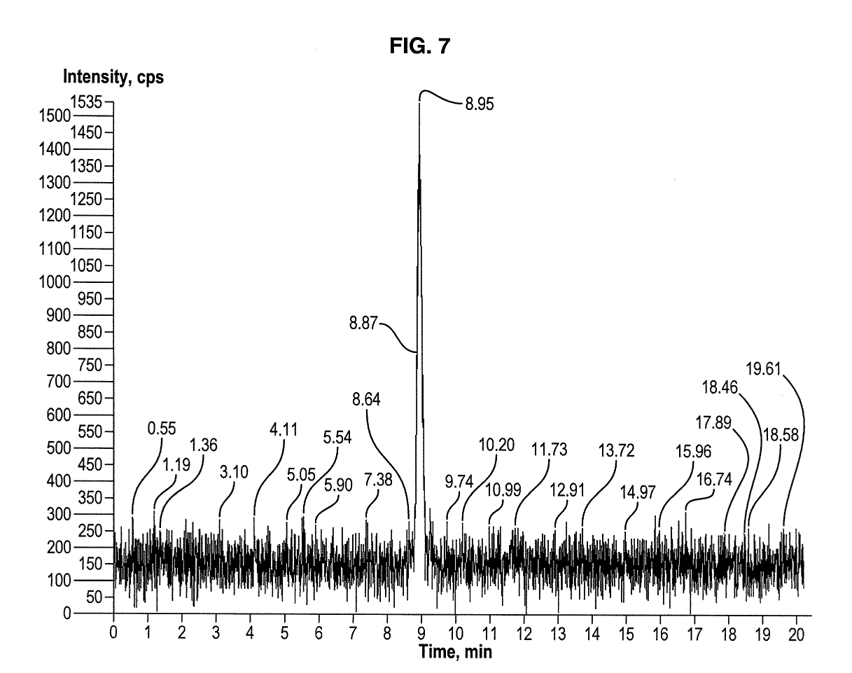

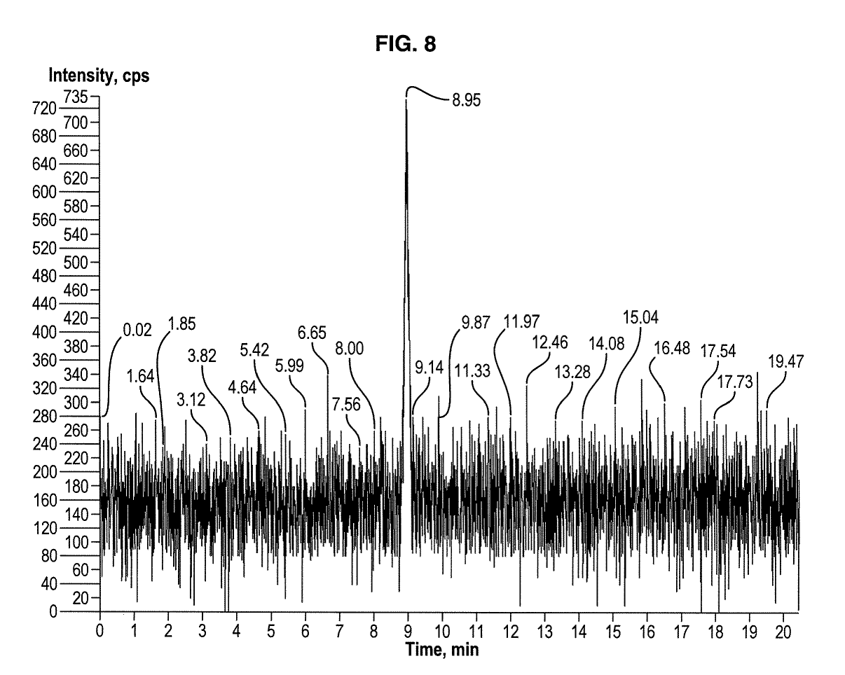

The results are showed on FIG. 7 or 8.

FIG. 7: reaction done with 100 mM of glycolate and 40 μg of crude cell-extracts of the strain AG1354 overproducing the protein PrepEst.

FIG. 8: reaction done with 40 mM of glycolate and 9 μg of the purified protein.

In each case, only one pick is detected with a mass of 825 (Mass-1=824.0) corresponding to the glycolyl-CoA.

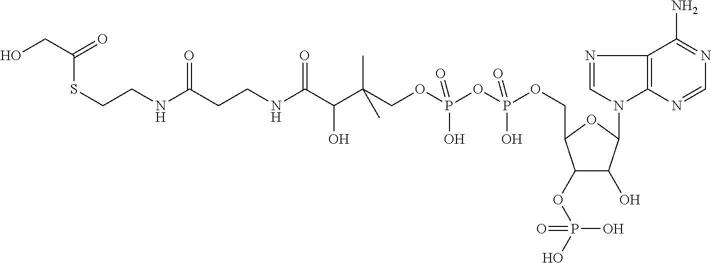

Molecular Weight=825.58

Exact Mass=825

Molecular Formula=C23H38N7O18P3S

### REFERENCES

### U.S. Patent Documents

U.S. Pat. No. 2,772,268 A November 2007 Cho J-H. et al.
U.S. Pat. No. 6,143,952 A November 2000 Srienc F. et al.

### International Patent Documents

WO 2004/038030 A2 May 2004 Martin D. et al.
WO 2007/141316 A December 2007 Soucaille P.
WO 2007/140816 A December 2007 Soucaille P.
WO 01/55436 August 2001 Green P. R.
WO 98/54329 December 1998 Witholt B. et al.
WO 99/61624 December 1999 Skraly F. et al.

### Other Publications

Williams and Peoples, CHEMTECH 26, 38-44, 1996,
Langer, R., Drug delivery and targeting. Nature 392, 5-10 (1998),
Ikada, Y. & Tsuji, H. Macromol. Rapid Commum. 21, 117-132, 2000,
Athanasiou, K. A. et al. Biomaterials 17, 93-102, 1996,
Lu, L. & Mikos, A. G. Poly (glycolic acid) in polymer data handbook (ed. Mark, J. E.) 566-569, Oxford University Press, New York, 1999,
Sorensen, W. R. & Campbell, T. W. Preparative Methods of Polymer Chemistry 2^{nd} edition, Interscience Publishers Inc, New York, 1963,
Dechy-Cabaret, O. et al. Chem. Rev. 104, 6147-6176, 2004,
Hyon, S.-H. et al. Biomaterials, 18, 1503-1508, 1997,
Lenz R. W. and Marchessault R. H. Biomacromolecules. 6:1-8, 2005
Lenz R. W. Adv Polym Sci 107:1-40, 1993
Sudesh K. et al. Progr Polym Sci 25:1503-1555, 2000
Sudesh K. and Doi Y. Handbook of biodegradable polymers. Rapra technology, UK, pp 219-256, (Bastioli C. Editions), 2005
Steinbüchel A. and Füchtenbusch B. TIBTECH 16:419-427, 1998
Steinbüchel A. Valentin A E. FEMS Microbiol Lett 1995, 128:219-228
Steinbüchel A. Biomaterials. Macmillan, New York, pp 123-213, Byrom D. Editions, 1991
Timm A and Steinbüchel A. Appl. Microbiol. Biotechnol. 56:3360-3367, 1990
Nomura, C. T. & Taguchi, S. Appl Microbiol Biotechnol. 73:969-79, 2007
Hazer B. & Steinbüchel A. Appl Microbiol Biotechnol. 74:1-12, 2007
Madison L. and Huisman G. Microbiol. and Mol Biol Reviews, 63:21-53, 1999
Rehm B. Biochem J. 376:15-33, 2003
Gerngross T. and Martin D. Proc Natl Acad Sci USA. 92:6279-83, 1995
Qi et al. FEMS Microbiol. Lett., 157:155, 1997
Qi et al. FEMS Microbiol. Lett., 167:89, 1998
Langenbach et al. FEMS Microbiol. Lett., 150:303, 1997
Amara A. et al. Appl Microbiol Biotechnol. 59:477-82, 2002
Fukui et al. J. Bacteriol. 180:667, 1998
Tsage et al. FEMS Microbiol. Lett. 184:193, 2000
Taguchi et al. FEMS Microbiol. Lett. 176:183, 1999
Ren et al., J. Bacteriol. 182:2978, 2000
Park et al, FEMS Microbiol. Lett. 214:217, 2002
Zhan et al. Appl. Microbiol. Biotechnol. 56:131, 2001
Valentin and Steinbüchel, Appl. Microbiol. Biotechnol. 40:699, 1994
Yuan et al., Arch. Biochem. Biophysics. 394:87, 2001
Valentin et al. Appl Environ Microbiol. 66:5253-8, 2000
Lin H. et al. Appl Microbiol Biotechnol. 71:870-4, 2006
Mack, M. and Buckel, W. FEBS Lett. 405:209-12, 1997
Sambrook et al. Molecular Cloning: a Laboratory Manual. 2^{nd} ed. Cold Spring Harbor Lab., Cold Spring Harbor, N.Y. 1989
Carrier et al. Biotechnol Bioeng. 59:666-72, 1998
Wieczorek R. et al. J. Bacteriol. 177: 2425-2435, 1995
York G. M. et al. J. Bacteriol. 184: 59-66, 2002

1. A method for obtaining the polymerisation of glycolic acid into PGA with a microorganism, comprising the steps of:
cultivating a microorganism expressing a gene encoding for an heterologous polyhydroxyalkanoate (PHA) synthase, in a medium comprising a carbon source,
and recovering the polymerised glycolic acid (PGA),
wherein the microorganism also expresses at least one gene encoding for an enzyme(s) transforming the glycolic acid into glycolyl CoA.

2. The method of claim 1 wherein the glycolic acid is produced by the same microorganism expressing genes encoding a PHA synthase and at least one enzyme transforming the glycolic acid into glycolyl CoA.

3. The method of claim 1 wherein the glycolic acid is provided to the microorganism exogenously in the culture medium.

4. The method of claim 1 wherein the transformation of the glycolic acid into glycolyl-coA is performed by at least one enzyme chosen among
a. acyl-CoA synthetases
b. acyl-CoA transferases, and
c. Phosphotransbutyrylase associated to butyrate kinase.
d.

5. The method of claim 4 wherein the acyl-CoA synthetase and the acyl-coA transferase are encoded by the genes prpE or acs.

6. The method of claim 4 wherein the phosphotransbutyrylase is encoded by the gene ptb and the butyrate kinase is encoded by the gene buk.

7. The method of claim 5 wherein said genes are overexpressed.

8. The method of claim 7 wherein said genes are expressed by a plasmid introduced into the microorganism.

9. The method of claim 7 wherein said genes have been integrated into the chromosome of said microorganism.

10. The method of claim 1 wherein the gene encoding a heterologous PHA synthase is chosen among phaC, phaEC and phaCR.

11. The method of claim 10 wherein said gene is overexpressed.

12. The method of claim 11 wherein said gene is expressed by a plasmid introduced into the microorganism.

13. The method of claim 11 wherein said gene has been integrated into the chromosome of said microorganism.

14. The method of claim 1 wherein the microorganism expresses the PhaR/PhaP regulatory system.

15. A polymerised glycolic acid obtained by the method according to claim 1.

16. A microorganism expressing genes encoding for an heterologous PHA synthase and at least one enzyme transforming the glycolic acid into glycolyl CoA as defined in claim 1.

17. The microorganism and enzyme of claim 16, wherein said microorganism is an Enterobacteriaceae.

18. The method of claim 5 wherein the gene prpE is from E. coli or from S. thyphimurium.

19. The method of claim 5 wherein said genes are overexpressed.

20. The method of claim 19 wherein said genes are expressed by a plasmid introduced into the microorganism.

21. The method of claim 19 wherein said genes have been integrated into the chromosome of said microorganism.

22. The microorganism and enzyme of claim 17, wherein said microorganism is an Escherichia coli.

23. The microorganism and enzyme of claim 16, wherein the enzyme transforming the glycolic acid into glycolyl-coA is chosen among the group consisting of:
a. acyl-CoA synthetases
b. acyl-CoA transferases, and
c. Phosphotransbutyrylase associated to butyrate kinase.

24. The microorganism and enzyme of claim 23 wherein the acyl-CoA synthetase and the acyl-coA transferase are encoded by the genes prpE or acs.

25. The microorganism and enzyme of claim 23 wherein the phosphotransbutyrylase is encoded by the gene ptb and the butyrate kinase is encoded by the gene buk.

26. The microorganism and enzyme of claim 16 wherein the gene encoding a heterologous PHA synthase is chosen among phaC, phaEC and phaCR.


SEQUENCE LISTING (9 sequences)

SEQ ID NO: 1 - DNA, 35 bp, Artificial Sequence
  Oligonucleotide
tctagaggatccaagttcaacaggagagcattatg

SEQ ID NO: 2 - DNA, 44 bp, Artificial Sequence
  Oligonucleotide
ggatccgctagccctaggtacgtactactcttccatcgcctggc

SEQ ID NO: 3 - DNA, 69 bp, Artificial Sequence
  Oligonucleotide
cgatttaattaatctagaggcgtaagttctaaggaggtatattatgtcttttagcgaattttatcagcg

SEQ ID NO: 4 - DNA, 37 bp, Artificial Sequence
  Oligonucleotide
tacgcctagggctagcctattcttcgatcgcctggcg

SEQ ID NO: 5 - DNA, 35 bp, Artificial Sequence
  Oligonucleotide
tctagaagatctcctacaaggagaacaaaagcatg

SEQ ID NO: 6 - DNA, 44 bp, Artificial Sequence
  Oligonucleotide
agatctgctagccctaggtacgtattacgatggcatcgcgatag

SEQ ID NO: 7 - DNA, 82 bp, Artificial Sequence
  Ptrc01 promoter
gagctgttgacaattaatcatccggctcgtataatgtgtggaattgtgagcggataacaatttacgtataaggaggtatatt

SEQ ID NO: 8 - DNA, 28 bp, Artificial Sequence
  Oligonucleotide
cgaattcctattcttcgatcgcctggcg

SEQ ID NO: 9 - DNA, 37 bp, Artificial Sequence
  Oligonucleotide
cccaagctttgatgtcttttagcgaattttatcagcg